(19) United States
(12) Patent Application Publication
(10) Pub. No.: US 2020/0352482 A1
(43) Pub. Date: Nov. 12, 2020

(54) DEVICE FOR NON-INVASIVELY MEASURING GLUCOSE CONCENTRATION

(71) Applicant: A.D. Integrity Applications Ltd., Ashkelon (IL)

(72) Inventors: Avner Gal, Hertzeliya (IL); Alexander M. Raykhman, East Greenwich, RI (US); Eugene Naidis, Ashkelon (IL); Yulia Mayzel, Beer-Sheva (IL); Alexander Klionsky, Ashdod (IL); Anatoly Diber, Ashkelon (IL)

(21) Appl. No.: 16/938,327

(22) Filed: Jul. 24, 2020

Related U.S. Application Data
(63) Continuation-in-part of application No. 13/540,656, filed on Jul. 3, 2012.
Application No. 13/540,656 is a continuation of application No. 13/090,535, filed on Apr. 20, 2011, now Pat. No. 8,235,897.
(60) Provisional application No. 61/328,344, filed on Apr. 27, 2010.

(51) Int. Cl. A61B 5/145; A61B 5/00

(52) U.S. Cl. CPC A61B 5/14532; A61B 5/6816

(57) ABSTRACT

Examples disclosed herein provide a combination of measurement channels each having a pair of measuring sub-channels. Each sub-channel measures the glucose concentration by monitoring a physical variable dependent on the glucose concentration in the subject's tissue. The subchannels of each measurement channel are orthogonal towards a common disturbance acting on each subchannel of the apparatus. Ultrasonic, electromagnetic, and thermal channels may be implemented. The non-invasive glucose monitor comprises a processing unit, which drives these sub-channels' sensors. The sensors may be located on a sensor unit configured as an ear clip. The sensor unit may include ultrasonic piezo transducers positioned on opposing portions of the ear clip and thus configured to be on opposite sides of the ear lobe, capacitor plates positioned on opposing portions of the ear clip, and a heater and a sensor positioned on the ear clip in close juxtaposition to the ear lobe.

### CROSS-REFERENCE TO RELATED APPLICATIONS

This application is a continuation-in-part of, and claims priority under 35 U.S.C. § 120 to, U.S. application Ser. No. 13/540,656, titled “DEVICE FOR NON-INVASIVELY MEASURING GLUCOSE,” filed on Jul. 3, 2012, which is a continuation of, and claims priority under 35 U.S.C. § 120 to, U.S. application Ser. No. 13/090,535, filed on Apr. 20, 2011, “DEVICE FOR NON-INVASIVELY MEASURING GLUCOSE,” which claims priority under 35 U.S.C. § 119(e) to U.S. Provisional No. 61/328,344, filed Apr. 27, 2010, titled “DEVICE FOR NON-INVASIVELY MEASURING GLUCOSE,” each of which is hereby incorporated by reference in its entirety.

### FIELD OF THE INVENTION

This invention relates to the medical field and the treatment of specified diseases and, in particular, to a device for non-invasive measurement of the glucose concentration of a subject patient.

### BACKGROUND OF THE INVENTION

Diabetes and its complications impose significant economic consequences on individuals, families, health systems and countries. The annual expenditure for diabetes in 2007 in the USA alone was estimated to be over $170 billion, attributed to both direct and indirect costs (American Diabetes Association. Economic costs of diabetes in the U.S. in 2007. Diabetes Care. 2008 March, 31(3): 1-20). In 2010, Healthcare expenditures on diabetes are expected to account for 11.6% of the total worldwide healthcare expenditure. It is estimated that approximately 285 million people around the globe will have diabetes in 2010, representing 6.6% of the world's adult population, with a prediction for 438 million by 2030 (International Diabetes Federation. Diabetes Atlas, Fourth edition. International Diabetes Federation, 2009).

In the recent years, research has conclusively shown that improved glucose control reduces the long-term complications of diabetes (DCCT Research Group. The effect of intensive treatment of diabetes on the development and progression of long-term complications in insulin-dependent diabetes mellitus. North England Journal of Medicine. 1993 Sep. 30; 329(14): 977-986; UKPDS Group: Intensive blood-glucose control with sulphonylureas or insulin compared with conventional treatment and risk of complications in subjects with type 2 diabetes (UKPDS33). The Lancet. 1998 Sep. 12; 352(9131): 837-853). According to the American Diabetes Association (ADA), self-monitoring of blood glucose (SMBG) has a positive impact on the outcome of therapy with insulin, oral agents, and medical nutrition (American Diabetes Association. Clinical Practice Recommendations, Standards of medical care in diabetes. Diabetes Care. 2006 Jan. 29: S4-S42). In its publication “Consensus Statement: A European Perspective”, the Diabetes Research Institute in Munich recommends SMBG for all types of diabetes treatment approaches, in order to achieve proper glucose control and values which are close to normal, without increasing the risk of hypoglycemia (Schnell O et al., Diabetes, Stoffwechsel and Herz, 2009; 4:285-289). Furthermore, special guidelines with proper recommendations were issued recently by the International Diabetes Federation (IDF), to support SMBG for non-insulin treated T2DM patients (Recommendations based on a workshop of the International Diabetes Federation Clinical Guidelines Taskforce in collaboration with the SMBG International Working group. Guidelines on Self-Monitoring of Blood Glucose in Non-Insulin Treated Type 2 Diabetics. International Diabetes Federation, 2009).

SMBG presents several benefits in both diabetes education and treatment. It can help facilitate individuals' diabetes management by providing an instrument for objective feedback on the impact of daily lifestyle habits, individual glucose profiles, including exercise and food intake impact on that profile, and thereby empower the individual to make necessary changes. Moreover, SMBG can support the healthcare team in providing individually tailored advice about lifestyle components and blood glucose concentration lowering medications, thus helping to achieve specific glycemic goals.

The IDF Diabetes Atlas Ninth edition 2019 provides the latest figures, information, and projections on diabetes worldwide. In 2019, Approximately 463 million adults (20-79 years) were living with diabetes; by 2045 this will rise to 700 million. The proportion of people with type 2 diabetes is increasing in most countries and 79% of adults with diabetes were living in low- and middle-income countries. 1 in 5 of people who are above 65 years old have diabetes. 1 in 2 (232 million) people with diabetes were undiagnosed. Diabetes caused 4.2 million deaths in 2019. Diabetes caused at least 760 billion US dollars in health expenditure in 2019—10% of total health care spending on adults in the United States. More than 1.1 million children and adolescents are living with type 1 diabetes. More than 20 million live births (1 in 6 live births) are affected by diabetes during pregnancy. 374 million people are at increased risk of developing type 2 diabetes.

The inconvenience, expenses, pain, and complexity involved in conventional (invasive) SMBG, however, leads to its underutilization, mainly in people with type 2 diabetes (Mollema E D, Snoek F J, Heine R J, Van der Ploeg H M. Phobia of self-injecting and self-testing in insulin treated diabetes patients: Opportunities for screening. Diabet Med. 2001; 18:671-674; Davidson M B, Castellanos M, Kain D, Duran P. The effect of self-monitoring of blood glucose concentrations on glycated hemoglobin concentrations in diabetic patients not taking insulin: a blinded, randomized trial. Am J Med. 2005; 118(4):422-425; Hall R F, Joseph D H, Schwartz-Barcott D: Overcoming obstacles to behavior change in diabetes self-management. Diabetes Educ. 2003; 29:303-311). Availability of an accurate, painless, inexpensive, and easy-to-operate device will encourage more frequent testing (Wagner J, Malchoff C, Abbott G. Invasiveness as a Barrier to Self-Monitoring of Blood Glucose in Diabetes. Diabetes Technology & Therapeutics. 2005 August; 7(4): 612-619; Soumerai S B, Mah C, Zhan F, Adams A, Baron M, Fajtova V, Ross-Degnan D. Effects of health maintenance organization coverage of self-monitoring devices on diabetes self-care and glycemic control. Arch Intern Med. 2004; 164:645-652), leading to tighter glucose control and the delay or decrease of long-term complications and their associated healthcare costs. Non-invasive (NI) glucose monitoring can decrease the cost of SMBG and increase meaningfully the frequency of testing. The main concern in NI methods is to achieve industry-acceptable results, despite the fact that no direct blood measurement is performed.

As is well known in the medical arts, one of the more important blood components to measure for diagnostic purposes is glucose, especially for diabetic patients. The well-known and typical technique for determining blood glucose concentration is to secure a blood sample and apply that blood to an enzymatically medicated colorimetric strip or an electrochemical probe. Generally, this is accomplished from a finger prick. For diabetic patients who may need to measure blood glucose a few times a day, it can immediately be appreciated that this procedure causes a great deal of discomfort, considerable irritation to the skin and, particularly, the finger being pricked, and, of course, increases a risk of infection.

For many years, there have been a number of procedures for monitoring and measuring the glucose concentration in humans and animals. These methods, however, generally involve invasive techniques and, thus, have some degree of risk, or at least some discomfort, to the patient. Recently, some non-invasive procedures have been developed, but they still do not always provide optimum measurements of the glucose concentration. At present, there has been no practical confirmed solution.

Most non-invasive monitoring techniques have focused on using incident radiation, which is capable of penetrating tissue and probing the blood. Currently known approaches to non-invasive glucose measurement are mainly based on optical technology. The less successful and relatively uncommon electrical measurements focus upon the dielectric properties of water solutions in a given frequency range, typically between 1-50 MHz. In one form or another, such methods attempt to monitor the influence of glucose or other analyzed concentration upon the dielectric frequency response of either the glucose itself or the secondary effect on the water.

Although investigations have been made into the use of acoustic monitoring, past studies have been primarily directed to the differences in acoustic velocity between organs. These studies have attempted to correlate acoustic velocity changes with chronic or continuous disease states. In addition, there is a large body of medical and scientific literature pertaining to the use of acoustic absorptive and scattering properties of organs for imaging, therapeutic and even diagnostic objectives.

A measuring instrument's performance is evaluated by statistical variables including accuracy and precision, and also by variables characterizing the instrument's trending pattern regardless of which variable is being measured.

Various studies, including studies identified above and other, similar studies, establish that a proven method of improving the precision of a measurement includes “smoothing.” Smoothing includes the application of some variant of averaging to a set of measurements produced by a measuring instrument. Increasing the number of individual measurements used by the averaging procedure yields a “smoother,” or less-disturbed, final sequence of readings generated by the instrument if the smoothing is performed under the conditions of a stationary system. From a statistical point of view, smoothing can be interpreted as a way to increase the confidence level of a representative sample of measurements.

There are at least two useful ways of developing the dataset used in such smoothing. One way requires collecting measurements separated by some time periods. Another way requires collecting measurements at a given moment of time from multiple measuring channels (MCs) where each MC demonstrates its affinity with the measured variable. For example, a measured variable may be a blood glucose concentration (BG). A combination of the above techniques enables a size of a representative sample to be substantially increased within a limited length of a time window, and simultaneously improves the confidence level of the representative sample. For example, a smoothed measured variable may be expressed as,

$$\overline{u ( t )} = {m}^{- 1} {\sum }_{i = 1}^{m} {n}^{- 1} {\sum }_{j = 1}^{n} {x}_{j , i} , \qquad ( 1 )$$

Here, u̅(̅t̅)̅ denotes the smoothed measured variable, x_{j,i} denotes the i^{th} instance of the measured variable value generated by the j^{th} MC before the smoothing procedure has been performed, n denotes the number of MCs in the measuring instrument, and m denotes the number of times the measurement was performed by each MC within a time window of a moving average procedure. U.S. Pat. No. 6,954,662 to Freger et al. (“Freger”) discloses a method of monitoring glucose using multiple measurement channels. FIG. 1 illustrates a table depicting a simplest numerical example of hypothetical readings from three MCs with identical measurement precision measuring a constant BG value of 100 mg/dL and exemplifies the value of the method of Freger. Columns MC1, MC2, MC3 of FIG. 1 are the readings from the three glucose measuring channels in units of mg/dL. According to the principles of measuring devices' uncertainty evaluation—for example, as described in Uncertainty of Measurement: A Review of the Rules for Calculating Uncertainty Components through Functional Relationships; NIST Technical Note 1297, 1994 Edition: Guidelines for Evaluating and Expressing the Uncertainty of NIST Measurement Results—the precision of the measurement can be expressed through the standard deviation of the measuring device's output, or its variance, or the coefficient of variance if the relative form is preferred:

$$P ∼ \frac{σ}{μ} \qquad ( 2 )$$

Where P denotes the precision of the measuring channel working under the stationary conditions, σ denotes the standard deviation of the measuring channel's output, and μ denotes the measuring channel's output sample mean. Cells PrcMC1, PrcMC2, and PrcMC3 of FIG. 1 denote the precision of each measurement channel as described in the expression (2), and DevPrc denotes the precision of the measuring device comprised of the above measurement channels. With the assumption that the informative or estimating variables of the above measuring channels can be approximated by a normally distributed random variable of the same mean and standard deviation, one can state that the precision of Freger's BG measuring device utilizing a linear combination of the three measuring channels algorithm will be smaller by a factor of \sqrt{3} as it follows from the Central Limit Theorem, as understood by one of ordinary skill in the art. Freger also discloses algorithmic solutions to further improve the performance of the BG measurement based on the assumption that all measuring channels are trending the BG correctly and that the rate of change of the BG is limited. These algorithmic solutions do not require changes in the construction of the measurement channels and are implementable within the computing means of the apparatus.

In the prior art apparatuses, a single MC always was used to measure the BG, thereby limiting the potential for measuring with higher precision and at a higher data update rate.

Freger discloses non-invasive methods (but not devices) for measurements of the speed of sound traveling through a subject's tissue, the electrical conductivity of the subject's tissue, and the heat capacity of the subject's tissue where the glucose is present in the subject's blood cells and in the interstitium. Thereafter, the glucose concentration for each of the three measurements is calculated and the final glucose value is determined by a weighted average of the three calculated glucose values.

While Freger mentions that measurements may be taken of the speed of sound through the tissue, the electrical conductivity of the tissue, and the heat capacity of the tissue, there is no disclosure of how any device can be constructed for effecting such measurements. Also, it is important to understand that there could be other BG-related variables of electrical, acoustic, and thermal origin that can be used to implement algorithms of Freger's method. Therefore, as used herein, a measurement channel producing the BG measurement by monitoring any physical variable related to the acoustic-wave propagation through the subject's tissue will be labeled the Ultrasonic Measurement Channel (UMC). A measurement channel producing the BG measurement by monitoring any physical variable related to the alternating electrical current flowing through media including the subject's tissue will be labeled the Electromagnetic Measurement Channel (EMC). A measurement channel producing the BG measurement by monitoring any physical variable related to a heatwave propagation through media including the subject's tissue will be labeled the Thermodynamic Measurement Channel (TMC).

Although the tissue's speed of sound propagation, the tissue's electrical conductivity, and the tissue's heat capacity were disclosed by Freger, various improvements to the BG measurement performance with respect to precision, accuracy, and long-term stability are disclosed herein by considering a variable commonly affecting each MC. For example, a common disturbance (that is, an error-inducing variable) affecting each MC of the apparatus of the present disclose may be the ambient temperature. A pair of two MCs out of three available MCs may be identified where the outputs of the MCs comprising the pair will trend in opposite directions with regards to the direction of change of the ambient temperature. That is, measurements generated by the two MCs exhibit “orthogonality” with respect to one another inasmuch as, while ambient temperature increases, measurements from a first MC of the two MCs will increase as a result of the ambient temperature increasing and measurements from a second MC of the two MCs will decrease as a result of the ambient temperature increasing. In this example, the ambient temperature induces a “positive error” with respect to an output of the first MC, or induces an error in a “positive direction,” inasmuch as ambient temperature causes the measurements of the first MC to increase (for example, erroneously past an actual target value), thereby inducing an error that may overshoot a target value. Similarly, as ambient temperature decreases, measurements from the second MC of the two MCs will increase as a result of the ambient temperature decreasing and measurements from the first MC of the two MCs will decrease as a result of the ambient temperature decreasing. In this example, the ambient temperature induces a “negative error” with respect to an output of the second MC, or induces an error in a “negative direction,” inasmuch as ambient temperature causes the measurements from the second MC to decrease (for example, erroneously below an actual target value), thereby inducing an error that may undershoot a target value. A pair of subchannels may thus be considered “orthogonal” where a positive error is induced in one subchannel and a negative error is induced in the other subchannel, at least because a disturbance variable, such as ambient temperature, induces an error in opposite directions from the target value. Improved methods for processing acquired data is further disclosed to further improve measurement performance. The herein disclosed and claimed invention, therefore, is an improvement of Freger and discloses a specific apparatus in which these measurements can be effected.

A typical performance of the known invasive and minimally invasive devices for the spot measurement of the BG is characterized by the Mean Absolute Relative Difference (MARD) variable and is between 7 to 10% Full Scale when the measurement range includes high values of the BG. The MARD value for the BG measurement at low BG, down from 100 mg/dL, is required to be no larger than 15 mg/dL by ISO 15197:2013. However, it may be beneficial for non-invasive glucose meters to further reduce measurement errors below the maximum allowable MARD value at least because reducing measurement errors can mitigate or avoid health complications associated with inaccurate blood glucose concentration measurements.

Reducing measurement errors may be particularly advantageous in non-invasive or minimally invasive blood glucose measuring devices. While certain existing non-invasive BG measuring devices may be intended only for supplementing use of invasive BG measuring devices—for example, because the non-invasive or minimally invasive BG measuring device yields measurement errors that are not acceptable—examples discussed herein yield measurement errors that are sufficiently low as to enable a standalone non-invasive or minimally invasive BG measuring device, that is, one that need not be supplemented by an invasive BG measuring device. Examples of non-invasive or minimally invasive BG measuring devices described herein may therefore replace invasive BG measuring devices in certain therapeutic situations where insulin dose adjustments are involved, at least because an accuracy of such example devices is sufficiently high as to enable the devices to be used as standalone devices.

Therefore, there is a need for a more accurate, more precise, non-drifting, and non-invasive device for measuring glucose concentration, which can be achieved by means of monitoring multiple physically different glucose-estimating variables. It is, therefore, an object of the present invention to provide a device for non-invasively measuring glucose concentration in a subject. These objects are achieved by the features of the claims and the following description, in particular by the following preferred aspects of the invention relating to preferred additional and/or alternative embodiments.

### SUMMARY OF THE INVENTION

This and other objects of the Invention are achieved by a device of the unitary or distributed structure, that is capable of non-invasively measuring the body's glucose concentration by a combination of readings from a multitude of measuring channels. Each measuring channel may include a pair of sub-measuring channels. Each sub-measuring channel of said pair produces the BG measurement by means of monitoring a distinct physical variable linked to the BG and possesses the orthogonality property toward a common disturbance when viewed from the point of output of said measuring channel. That is, the sub-measuring channels of the pair of sub-measuring channels produce measurements that are orthogonal with respect to one another relative to a common variable, which may include ambient temperature. The proposed information structure thus supports the BG measurements with greater performance than the performance of each individual component of said structure at least in part by utilizing the principle of orthogonality to offset an error produced in individual measurement channels.

One example of the disclosure includes three distinct sub-measuring channels.

In particular, one example device includes a sensory network and signal/data processing means. In some examples, a functional member may include the sensory network and signal/data processing means and can be implemented in a single module or separate communicated modules. One example includes a Processing Unit, containing hardware and software applications, and a Sensor Unit(s)/external device(s) (for example, an ear clip or other fastener) contained within a housing for affixing to the patient. That is, the housing may include the Sensor Unit(s) (and, consequently, one or more sensors within the Sensor Unit[s]) and one or more fasteners for affixing the Sensor Unit(s) to a patient or subject. The Sensor Unit includes a first member and a second member which are connected to each other, wherein the first member and the second member are located at opposing sides of a part of the subject, to which said Sensor Unit is affixed. For instance, when the Sensor Unit is affixed to a patient's ear lobe, the two opposing members are located on the two opposing sides of the ear lobe. In another example, said first and second members are located at the same side of the subject's tissue.

In some examples, the unitary Sensor Unit may be incorporated with at least one of three members, which effect three separate and distinct non-invasive measurements of glucose. Additionally, it is further preferred to provide at least two or three members to effect two or three separate and distinct non-invasive measurements of glucose, respectively. According to a preferred embodiment of the present invention, at least three different members to effect three separate and distinct non-invasive measurements of glucose are provided within a single, unitary external device, for example, within a single housing.

It should also be appreciated and understood that each of the measurement channels is new and novel in and of themselves. Hence each measurement channel may be used in isolation by itself (or with still other measurement channels). By combining the three measurement channels in one unitary device, measurements are obtained from three separate and unique measurement channels, thereby improving the precision of the apparatus.

For the non-invasive measurement by use of ultrasound, preferably a transmitter (such as an ultrasonic transmitter) and a receiver (such as an ultrasonic receiver) are mounted on opposing sides of the Sensor Unit. When the Sensor Unit is fitted on the patient, a portion of the patient's body (such as an ear lobe) is situated between the transmitter and receiver. Upon receipt of a resultant signal after the signal passes through the patient, the receiver sends the signal to the Processing Unit for processing by appropriate algorithms. In some embodiments, membranes may be used to cover and protect the transmitter and receiver.

To effect an electromagnetic measurement, a capacitor is defined in the Sensor Unit. The capacitor plates are positioned on opposing sides of the external device and the body part (such as an ear lobe) disposed between the parts of the Sensor Unit serves as the dielectric. In some cases, the membranes used to shield or cover the transmitter and receiver can serve also as the capacitor plates.

The third measurement channel is based on the thermodynamic technology to measure the glucose concentration. For this purpose, preferably a heater and a temperature sensor are provided at the external device. The heater and the temperature sensor may be provided at opposing sides of the external device. According to another example, however, it is preferred to mount the heater and the temperature sensor on the same side of one of the two opposing sides, for example, on the tip of one side of the Sensor Unit the heater and the temperature sensor are positioned.

The objects of the present invention are solved, for example, by the following aspects of the invention.

According to the first aspect, a unitary device for non-invasively measuring glucose concentration in a subject comprises: ultrasonic piezo transducers positioned on opposing members of the device and surrounding a part of the subject's body to which the device is attachable; capacitor plates positioned on opposing members of said device and surrounding said part of the subject's body to which the external means is attachable, a temperature sensor and a heater positioned in close juxtaposition to said part of the subject's body to which the device is attachable.

In one example, the device further comprises an external means (such as an ear clip) to be affixed to the subject's body, wherein the ultrasonic piezo transducers, the capacitor plates, the heater, and the temperature sensor are contained within said external means.

There may also be a Processing Unit for controlling measurements and calculating glucose concentration; and, means for electrically connecting the Processing Unit and the external means, either galvanically or wirelessly.

In some examples, membranes cover the ultrasonic piezo transducers.

The ultrasonic piezo transducers may include a transducer and a receiver.

In some examples, the capacitor plates comprise membranes. In such an example, the membranes may also cover the ultrasonic piezo transducers.

An embodiment may include means for determining a distance between opposing portions of said external means. In some examples, this means may include a magnet and a sensor.

There may also be an adjustment screw setting the distance between opposing portions of said external means.

In some examples, an ambient temperature sensor may be included.

According to other aspects, the individual measurements channels may be separately utilized.

According to a second aspect, a device for non-invasively measuring glucose concentration in a subject may comprise a housing; and, capacitor plates positioned on opposing portions of the housing and surrounding a part of the subject's body to which the device is attachable, and auto-oscillating means connected to the capacitor plates.

In another example, this device also includes a processing means for calculating the glucose concentration based on the tissue impedance and means for communicating the tissue impedance signal to the processing means.

This example may include capacitor plates comprised of membranes.

According to another example, there may also be ultrasonic piezo transducers positioned on opposing members of the housing and surrounding said part of the subject's body to which the device is attachable. It may include capacitor plates comprised of membranes and the membranes may cover the ultrasonic piezo transducers.

Another example may include ultrasonic piezo transducers positioned on opposing members of the housing and surrounding the part of the subject's body to which the device is attachable, means for detecting a phase shift between a transmitted and a received wave, and processing means for calculating glucose concentration based on the phase shift and being in communication with the means for detecting.

In some examples, there may also be a heater and a sensor positioned on the device in close juxtaposition to the part of the subject's body to which said device is attachable. It may include means for communicating heat transfer characteristics to the processing means for calculating the glucose concentration.

According to another example, a device, for non-invasively measuring glucose concentration affixed to a part of a subject's body, comprises ultrasonic piezo transducers positioned on opposing members of the device and surrounding a part of the subject's body to which the device is attachable; and means for detecting a phase shift between a transmitted and a received wave.

Some examples may include a processing means for calculating the glucose concentration based on said phase shift and being in communication with the means for detecting.

According to an alternate version of this example, there may also be a heater and a sensor positioned on the device in close juxtaposition to the part of the subject's body to which said device is attachable. It may include means for communicating heat transfer characteristics to the processing means for calculating the glucose concentration.

According to a fourth aspect, a device, for non-invasively measuring glucose concentration affixed to a part of a subject's body, comprises a heater and a sensor positioned on the device in close juxtaposition to the part of the subject's body to which the device is attachable; and means for communicating heat transfer characteristics to a processing means for calculating glucose concentration.

According to an aspect of the disclosure, an apparatus for non-invasively measuring a blood glucose concentration in a subject is provided, the apparatus comprising a plurality of sensors, each sensor of the plurality of sensors being configured to determine sensor information indicative of a respective physical variable of a plurality of physical variables, each respective physical variable being indicative of the blood glucose concentration in the subject, and a controller coupled to the plurality of sensors, the controller being configured to receive respective sensor information from each sensor of the plurality of sensors, determine at least one measurement channel each including an orthogonal pair of sensors from the plurality of sensors, each orthogonal pair of sensors including a first sensor to determine first sensor information indicative of the blood glucose concentration of the subject and a second sensor to determine second sensor information indicative of the blood glucose concentration of the subject, wherein the orthogonal pair of sensors is orthogonal with respect to a disturbance variable that induces a positive error in one of the first sensor information and the second sensor information and that induces a negative error in the other of the first sensor information and the second sensor information, and determine a blood glucose measurement of the subject based on the first sensor information and the second sensor information.

In some examples, the at least one orthogonal pair of sensors includes a single pair of sensors. In various examples, the disturbance variable includes an ambient temperature. In at least one example, the plurality of physical variables includes one or more of a property of an ultrasonic wave propagating through a tissue of the subject, a property of an electromagnetic impedance of the tissue of the subject, and a property of a heatwave propagating through the tissue of the subject. In some examples, the apparatus includes a housing including the plurality of sensors and a fastener configured to affix the housing to the subject. In various examples, the plurality of sensors includes a first ultrasonic piezo transducer and a second ultrasonic piezo transducer, and wherein the first ultrasonic piezo transducer and the second ultrasonic piezo transducer are configured to be coupled to opposing sides of a portion of a body of the subject.

In at least one example, the apparatus includes a respective membrane covering each of the first ultrasonic piezo transducer and the second ultrasonic piezo transducer. In some examples, at least one of the first ultrasonic piezo transducer includes an ultrasonic-wave transmitter and the second ultrasonic piezo transducer includes an ultrasonic-wave receiver, wherein the first ultrasonic piezo transducer is configured to transmit an ultrasonic wave to the body of the subject, and wherein the second ultrasonic piezo transducer is configured to receive the ultrasonic wave from the body of the subject. In various examples, the first sensor includes the first ultrasonic piezo transducer and the second ultrasonic piezo transducer, and wherein the first sensor information includes a phase shift between the transmitted ultrasonic wave and the received ultrasonic wave.

In at least one example, the plurality of sensors includes a first capacitor plate, a second capacitor plate, and an auto-oscillator configured to generate an oscillating signal between the first capacitor plate and the second capacitor plate, and wherein the first capacitor plate and the second capacitor plate are configured to be positioned on opposing sides of a portion of a body of the subject. In some examples, the apparatus includes a respective membrane covering each of the first capacitor plate and the second capacitor plate. In various examples, the first sensor includes the first capacitor plate and the second capacitor plate, and wherein the first sensor information includes a tissue impedance of the subject.

In at least one example, the plurality of sensors includes a heater and a heat sensor configured to be coupled to a portion of a body of the subject. In some examples, the first sensor includes the heater and the heat sensor, and the first sensor information includes heat transfer characteristics of the subject. In various examples, the fastener includes opposing sides configured to affix the housing to a body of the subject, the apparatus further comprising at least one distance sensor configured to measure a distance between the opposing sides of the fastener. In some examples, the at least one distance sensor includes a magnet and a magnetic-field sensor. In at least one example, the apparatus includes an adjustment screw configured to set the distance between the opposing sides of the fastener.

In at least one example, the at least one measuring channel includes a first measuring channel and a second measuring channel, the first measuring channel including a first orthogonal pair of sensors, the first orthogonal pair of sensors being configured to measure a first and a second of the property of the ultrasonic wave propagating through the tissue of the subject, the property of the electromagnetic impedance of the tissue of the subject, and the property of the heatwave propagating through the tissue of the subject, and the second orthogonal pair of sensors being configured to measure the first and a third of the property of the ultrasonic wave propagating through the tissue of the subject, the property of the electromagnetic impedance of the tissue of the subject, and the property of the heatwave propagating through the tissue of the subject.

According to at least one aspect, a method for non-invasively measuring a blood glucose concentration in a subject is provided comprising determining, with each sensor of a plurality of sensors, sensor information indicative of a respective physical variable of a plurality of physical variables, each respective physical variable being indicative of the blood glucose concentration in the subject, determining at least one measurement channel each including an orthogonal pair of sensors from the plurality of sensors, each orthogonal pair of sensors including a first sensor to determine first sensor information indicative of the blood glucose concentration of the subject and a second sensor to determine second sensor information indicative of the blood glucose concentration of the subject, wherein the orthogonal pair of sensors is orthogonal with respect to a disturbance variable that induces a positive error in one of the first sensor information and the second sensor information and that induces a negative error in the other of the first sensor information and the second sensor information, and determining a blood glucose measurement of the subject based on the first sensor information and the second sensor information.

According to at least one aspect of the disclosure, a system for non-invasively measuring a blood glucose concentration in a subject is provided comprising a plurality of sensors, each sensor of the plurality of sensors being configured to determine sensor information indicative of a respective physical variable of a plurality of physical variables, each respective physical variable being indicative of the blood glucose concentration in the subject, and means for determining a blood glucose measurement of the subject based on sensor information from at least one orthogonal pair of sensors more accurately and more precisely than any individual sensor of the at least one orthogonal pair of sensors.

Other objects, features and advantages of the present invention will become apparent upon reading the following detailed description in conjunction with the drawings and the claims.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings, which illustrate examples of embodiments of the invention, in which:

FIG. 1 illustrates a table depicting an example of readings from three measurement channels of a blood glucose concentration, and processed data produced based on the readings, according to an example;

FIG. 2 illustrates an information flow block diagram of a blood glucose measuring apparatus according to an example;

FIG. 3 illustrates a perspective view of a blood glucose measuring device including a Processing Unit (PU) and a personal ear clip (PEC) according to an example;

FIG. 4 illustrates a side cross-sectional view of the PEC according to an example;

FIG. 5 illustrates a view of a sensor-tissue structure for one embodiment of the Thermodynamic channel of measurement according to an example;

FIG. 6 illustrates a graph showing the non-temperature-corrected process of heating the sensor-tissue structure in a subject, reflecting different glucose concentrations according to an example;

FIG. 7 illustrates a graph showing an integrated and temperature-corrected equivalent thermal signal in a subject versus glucose concentration according to an example;

FIG. 8A illustrates a schematic representation of the earlobe between the two ultrasonic piezo transducers for the Ultra Sound channel of measurement according to an example;

FIG. 8B illustrates a graph showing the Phase shift between the received and transmitted waves, measured as Δφ according to an example;

FIG. 9 illustrates a graph showing the phase shift versus the transducer's frequency-of-excitation in the low-frequency region; and, the amplified phase-shift values are viewed at a chosen frequency value determined during a calibration procedure for a subject according to an example;

FIG. 10 illustrates a graph for a subject, in the Ultrasonic Measuring channel, showing a temperature-corrected phase shift measured at a chosen frequency versus glucose concentration for a subject in the Ultrasonic measuring channel according to an example;

FIG. 11 illustrates the Electromagnetic Measuring Channel's information block diagram according to an example;

FIG. 12 illustrates a graph showing a temperature-corrected Electromagntic signal frequency versus glucose concentration for a subject according to an example;

FIG. 13 illustrates a perspective view of the PEC according to an example;

FIG. 14 illustrates a side view of the PEC according to an example;

FIG. 15 illustrates a side cross-sectional view of the PEC according to an example;

FIG. 16A illustrates a perspective view of the members of the thermal channel according to an example;

FIG. 16B illustrates an end view, partially in section, of the members of an alternate embodiment of the thermal channel according to an example;

FIG. 16C illustrates an end view, partially in section, of the members of an alternate embodiment of the thermal channel according to an example;

FIG. 17 illustrates a side cross-sectional view of a first membrane for the ultrasonic transducer, which may also serve as one of the plates of the capacitor for the electromagnetic channel according to an example;

FIG. 18 illustrates a side cross-sectional view of a second membrane for the ultrasonic transducer, which may also serve as one of the plates of the capacitor for the electromagnetic channel according to an example;

FIG. 19A illustrates an enlarged side cross-sectional view of the tip of the PEC and showing the members constituting the measurement channels according to an example; and

FIG. 19B illustrates an enlarged top cross-sectional view of a portion of the tip of the PEC according to an example.

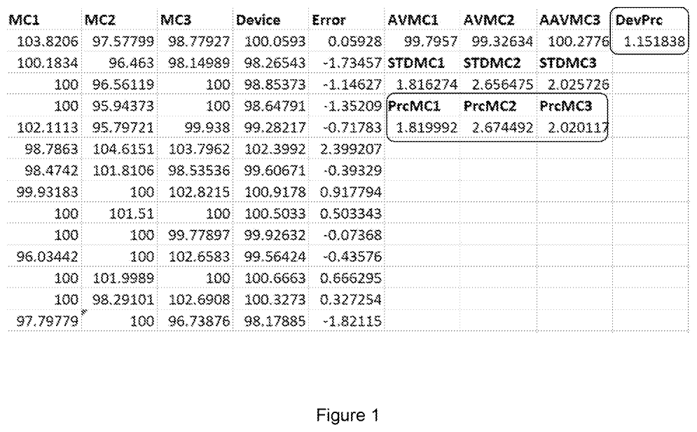

### DETAILED DESCRIPTION OF THE PREFERRED EMBODIMENT

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The preferred embodiment of the system and its advantages are best understood by referring to the drawings and the following description where like numerals indicate like and corresponding parts of the various drawings. References to preferred embodiments are for illustration and understanding and should not be taken as limiting.

While the herein description is with regard to a human patient, it may be appreciated that examples discussed herein can be used to measure glucose concentration in any subject, including animals.

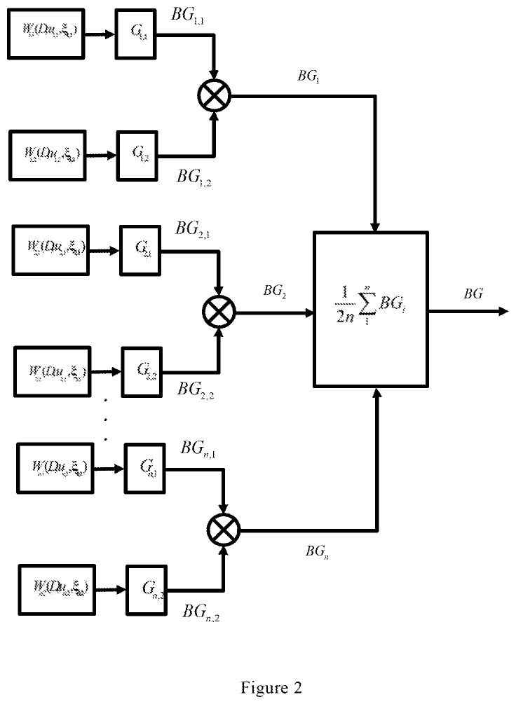

This and other objects of the disclose are achieved by a device of the unitary or multimodule construction enabling non-invasive measurement of the body's glucose concentration by a combination of readings from a multitude of measuring channels. From the information flow's point of view, each measuring channel is comprised of a pair of sub-measuring channels. For example, FIG. 2 illustrates an information flow block diagram of a blood glucose concentration determination scheme according to at least one example. A blood glucose concentration value BG is generated and outputted by summing a plurality of individual blood glucose concentration measurements BGᵢ, i=1̅,̅n̅ received from respective measuring channels. Each individual blood glucose concentration measurement BGᵢ is generated by a measurement channel having a pair of two sub-measuring channels. Each sub-measuring channel of said pair produces the BG measurement BG_{i,j}, j=1, 2 by means of monitoring a distinct physical variable linked to the BG and possessing the orthogonality property toward a common disturbance when viewed from the position of output of said measuring channel. That is, as a variable, such as ambient temperature, induces an error in a measurement value in one direction (for example, increasing the error as the variable increases) for one sub-channel in the pair of sub-channels, the variable also induces an error in a measurement value in a second, opposite direction (for example, decreasing the error as the variable increases) for the other sub-channel of the pair of sub-channels. The proposed information structure thus supports the BG measurements with greater performance than the performance of each individual component of said structure by collectively offsetting an error introduced by a variable such as ambient temperature.

As illustrated by FIG. 2, each sub-channel has a transfer operator

$${W}_{i , j} ( D , {u}_{i , j} , {ξ}_{i , j} ) , D ≡ \frac{d}{dt} ,$$

and a gain value G_{i,j}, where i=1̅,̅n̅; j=1, 2, to process data measured by the respective sub-channel, where the gain value may be implemented to assign weights to an associated variable; u_{i,j} denotes a subchannel's useful (relevant to BG) variable; ξ_{i,j} denotes a subchannel's vector of glucose-irrelevant variables (which may include error-inducing variables) including the sub-channel's disturbing variables, such as an ambient temperature.

It will be shown below in various examples herein that depending on an environment (for example, including respective types and strengths of disturbing variables), various parts of the proposed apparatuses' information structure can be implemented as separate BG measuring devices.

One example of the present invention includes three distinct measuring channels.

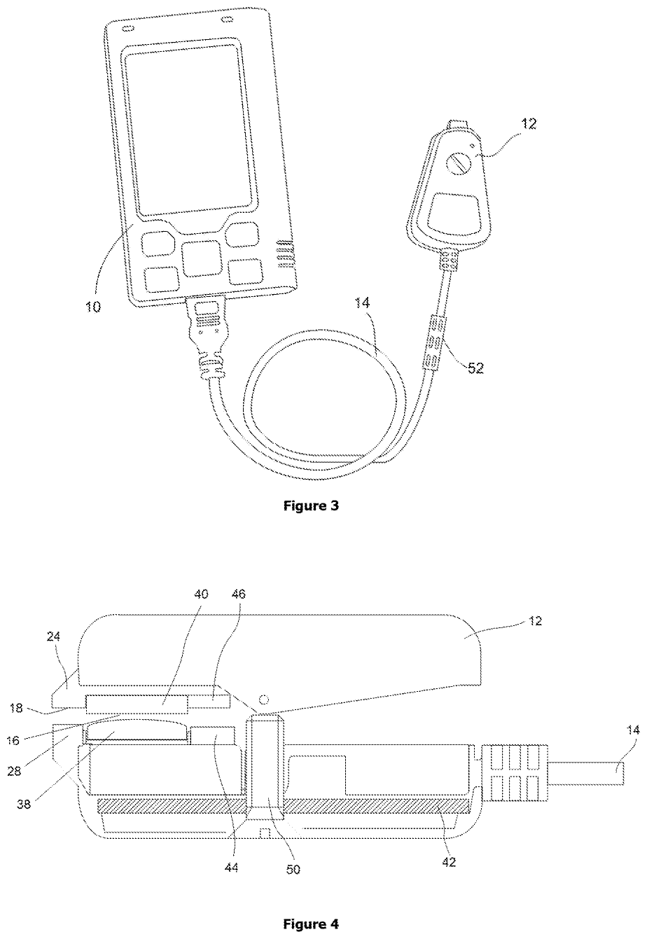

Constructively, as shown in the FIG. 3, the device includes a Processing Unit (PU) 10 containing the software applications and a Sensor Unit 12 for affixing to the patient. The Sensor Unit 12 may be placed on a subject's ear lobe, such that the Sensor Unit 12 may be configured as an ear clip in some examples.

A cable 14 is preferably used to provide a working connection between the PU 10 and the Sensor Unit 12. It may be appreciated that wireless (for example, Bluetooth) technology may also be used in lieu of, or in addition to, the cable 14.

It should be appreciated that the Sensor Unit 12 may be placed on any other suitable location of the subject's body, such as a toe, a finger, the web between the thumb and second finger (forefinger), and so forth. For example, the Sensor Unit 12 may be placed on a body part that has skin and tissue characteristics similar to those of the ear lobe. When the Sensor Unit is placed on the body at a point other than the ear lobe, some adjustment of the algorithms may be necessary, as the skin and tissue characteristics may not be uniform over the entire body.

Said device measures three values of the BG by means of three measuring channels. It is to be appreciated that, in some examples, a device may measure a different number of values of BG, and that three BG values are provided for purposes of example. These glucose values are employed to calculate the final BG value, which may be output as the device's reading. In order to increase the precision of the non-invasive glucose measurement, the device, according to the present invention, preferably uses a combination of more than one non-invasive method implemented within more than one measuring channel: ultrasonic, electromagnetic, and thermodynamic. These measuring channels utilize the tissue's physiological response to glucose variations, resulting in changes of physical properties such as electromagnetic and acoustic impedance, and heat transfer (HT) characteristics of the cellular, interstitial and plasma compartments, due to changes in ion concentration, density, compressibility, thermal diffusivity, and hydration of those compartments.

As shown in FIG. 3, this non-invasive glucose monitor comprises a PU 10, which drives a plurality of different sensor channels, such as three different sensor channels (preferably one per technology), located on the Sensor Unit 12 configured as a personal ear clip (PEC). In order to perform a spot measurement, the PEC is clipped externally to the user's earlobe for the duration of the measurement (about a minute) and is removed afterwards. A cable 14 (or any existing wireless [for example, Bluetooth] technology) connects these two components of the device.

One unique aspect of the invention is that the (single) Sensor Unit 12 houses more than one measuring channel. More preferably it houses all members to effect a plurality of separate and distinct non-invasive glucose measurements. Preferably, the Sensor Unit houses members to effect three separate and distinct non-invasive glucose measurements by three separate and distinct technologies. This single external device provides the advantage that only one single device has to be attached to the subject's body, which is convenient for a physician and/or a patient. In some examples the Sensor Unit 12 is configured as an ear clip, as illustrated in FIG. 4. Accordingly, as used herein, the “ear clip,” or “PEC,” may be an example of, include, or be included within, the Sensor Unit 12, and the terms ear clip or PEC may therefore be used interchangeably with the Sensor Unit 12 in some examples.

It should also be appreciated and understood that constructively each measuring channel is new and novel in and of themselves. Hence each measurement sub-channel may be used in isolation by itself (or in combination with other measuring channels). It will be further demonstrated that combining the three unique measuring channels in one unitary device creates a foundation for the generation of final measurements (readings) with higher accuracy and precision than of each component measuring channel and under the improved ergonomic conditions.

Thermodynamic Measuring Channel (TMC).

Blood glucose variations affect HT characteristics through changes of the heat capacity of the tissue due to water/electrolytes shifts, as discussed in Zhao Z. Pulsed Photoacoustic Techniques and Glucose Determination in Human Blood and Tissue. Acta Univ. Oul C 169. Oulu, Finland, 2002, density, as discussed in Toubal M, Asmani M, Radziszewski E, Nongaillard B. Acoustic measurement of compressibility and thermal expansion coefficient of erythrocytes. Phys Med Biol. 1999; 44:1277-1287, and thermal conductivity, as discussed in Muramatsu Y, Tagawa A, Kasai T. Thermal Conductivity of Several Liquid Foods. Food Sci. Technol. Res. 2005; 11(3):288-294, Hillier T A, Abbot R D, Barret E J. Hyponatremia: evaluating a correction factor for hyperglycemia. Am J Med. 1999 April; 106(4):399-403; Moran S M, R L Jamison. The variable hyponatremic response to hyperglycemia. West J Med. 1985 January; 142(1):49-53). Thus, changes in glucose concentration alter the heat transfer processes occurring in the multi-layer sensor-tissue mechanical structure, as discussed in Wissler E H. Pennes' 1948 paper revisited. J Appl Physiol. 1998 July; 85(1):35-41. The higher the glucose concentration, the higher the thermal diffusivity of the tissue, thus causing greater temperature elevation in the exterior tissue layers in response to heating. Since the sensor(s) (for example, thermistor[s]) of the Sensor Unit 12 is (are) preferably mounted/affixed on the epidermis layer of a subject, the measured rate and magnitude of the temperature change of the subject responsive to heating is greater than in the internal tissues.

The Thermodynamic method, according to the present invention, applies a specific amount of energy to the tissue. Preferably both the rate and/or the magnitude of the temperature change, caused by the application of the known amount of energy to the tissue, are functions of the heat capacity, density, and thermal conductivity of the tissue. Thus, the device according to the present invention provides means such that the glucose concentration is preferably evaluated indirectly by measuring changes in the HT characteristics, obtained after tissue heating for a predetermined duration of time.

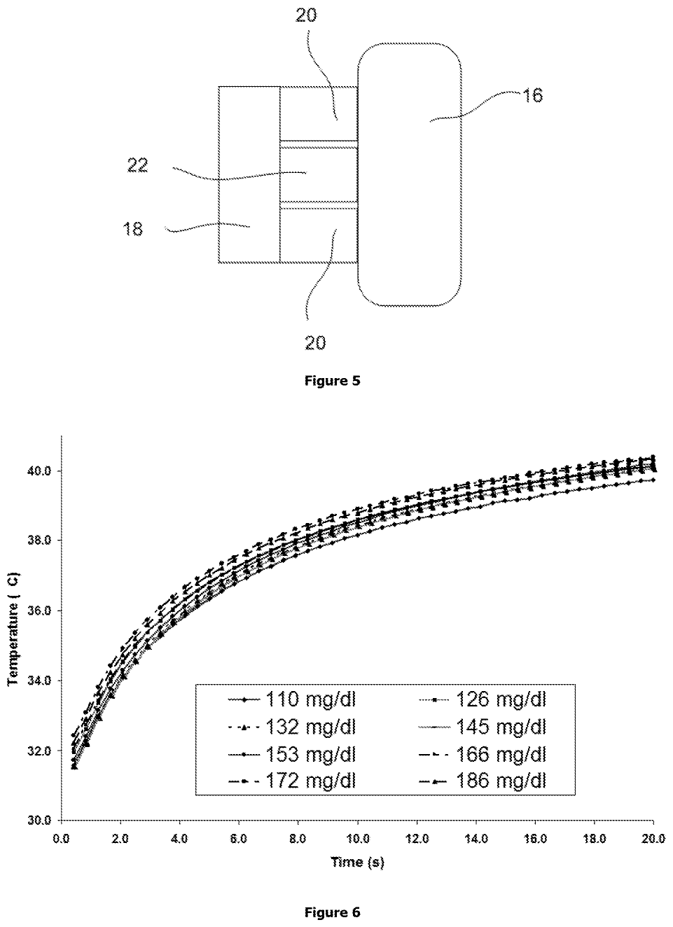

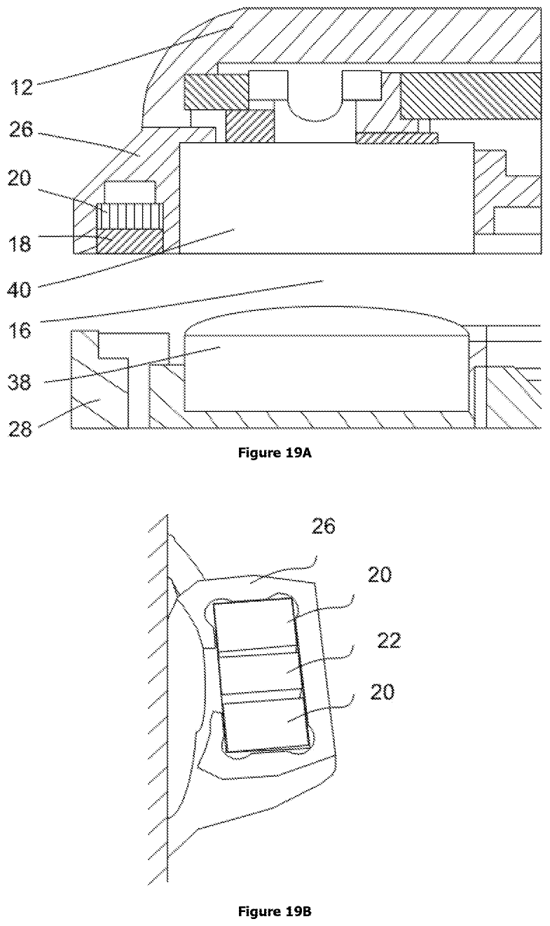

FIG. 5 shows a sensor-tissue structure, according to a preferred embodiment of the present invention. A bottom plate serves as a heater 18 and heat conductors 20 are included (see FIGS. 19A, 19B). A thermal sensor 22 is located in the middle between the conductors 20. As shown in FIG. 4, the thermal sensor is located on the tip 24 of the PEC.

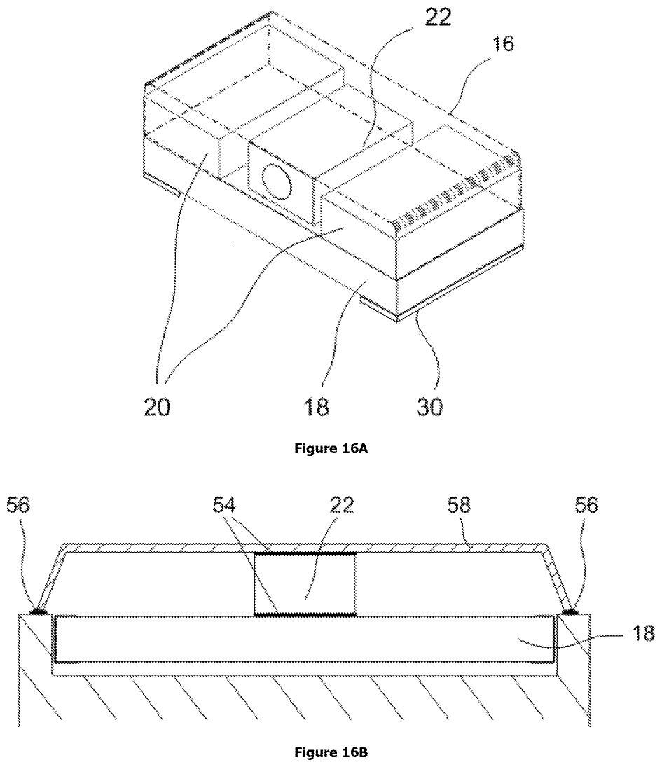

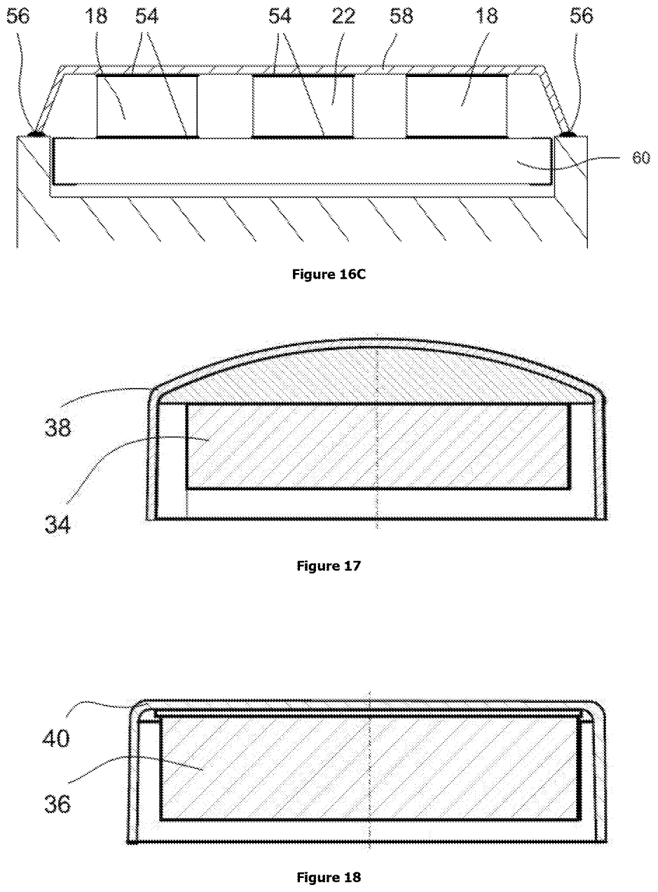

Referring to FIG. 16A, the thermistor module, which preferably comprises a plurality of thermistor components such as a thermal sensor 22, a heater 18 and conductors 20, is located on an ear 26 extending from the end of one side of the PEC (for example on the first portion of the PEC). The opposing surface 28 (that is, the second portion of the PEC) (see FIG. 19A) is preferably empty with no thermistor components. In other words, the heater 18 and the thermal sensor 22 may be located on the same side of the PEC. In particular, it is preferred that the heater 18 and the temperature sensor 22 are located on the same side with regard to an ear lobe, when the Sensor Unit 12 is attached to the ear lobe.

As depicted in FIGS. 16A, 16B, and 16C, each of which illustrates an example of a thermistor module (or thermal channel), the heater 18 may be made as a plate or block and is preferably constituted by a resistor. Two plates 20 are secured to the top of the plate to conduct heat energy and serve as conductors. This may be done by adhering, gluing, bonding, or any other suitable means. Preferably the plates 20 are aluminum, but any heat-conducting material may be used. On the bottom of the plate, preferably soldering pads 30 are provided which may be used to connect the heater 18 to integrated circuit boards 42 (see FIG. 15). Preferably, a housing contains all the sensor (for example, thermistor) modular components. In some examples, for a 4 V system, the resistor (for example, the heater plate) has a resistance between 23 and 43 Ohms and is preferably 33 Ohms. It produces temperatures in the range of about 15° to 45° C. and is preferably about 42-45° Centigrade. Any suitable heat sensor may be used. The heater sends heat energy into the earlobe. It begins the heating process at standard ambient temperature 15-35° C. Usually the ear lobe is a little warmer at 25-28° C. The power of the heater provides preferably a maximum of 0.5 W and preferably a minimum of 0.2 W. Usually the heater heats for 20 seconds. According to other preferred embodiments, however, a smaller heat energy may be used which preferably heat for longer times. Also, a larger heat energy may be used which preferably heat for a shorter time in other examples.

As may be appreciated, the thermistor module should be small enough to fit on the tip of the PEC. Preferably the resistor plate, constituting the heater 18, is about 5 mm long, 0.6 mm thick, and 2.4 mm wide. The conductors 20 are preferably 1.5 mm long, 0.7 mm thick, and 2.4 mm wide. As for the sensor 22, it is preferably 1.30 mm long, 0.8 mm thick, and 2.0 mm wide. These are standard parts available in the marketplace; and, hence the standard available sensor is not as wide as the resistor plate and conductors and extends slightly above the conductors. A small difference in the overall dimensions is not critical.

FIG. 6 depicts the time diagram of the non-temperature-corrected process of heating the sensor-tissue structure in a subject. The different curve shapes of the heating process represent different glucose concentrations.

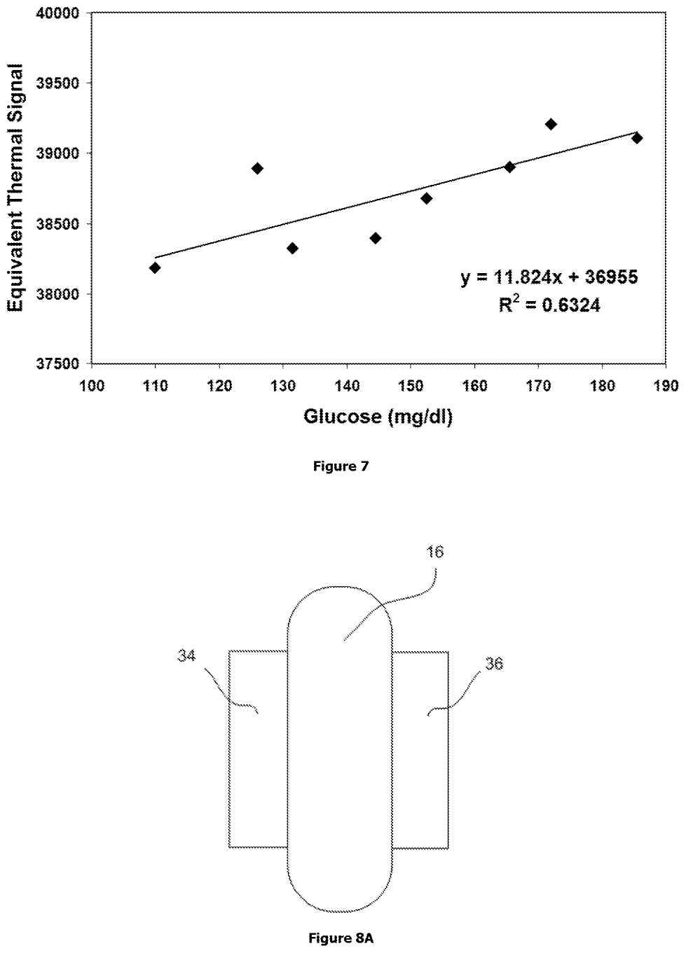

The ambient temperature that defines the boundary condition of the surface skin temperature and the sensor's initial temperature affects the process also. Therefore, the electrical signal u_{IMC}(t) reflecting the thermodynamic process is conditioned and normalized to consider the initial skin surface temperature, followed by the compensation for the difference between the ambient and skin temperatures. The integrated, temperature-corrected, and compensated signal û(t) is shown in the time diagram of FIG. 7, as a function of the glucose concentration. Furthermore, the signal û(t) may be expressed as,

$${u}_{TMC} ( t ) = {\int }_{{t}_{0}}^{{t}_{f}} F ( u ( t ) ) dt - {T}_{e} ( {t}_{f} - {t}_{0} ) - {q}_{TMC} ( {T}_{e} - {T}_{a} ) \qquad ( 3 )$$

where t₀ and t_{f} are the starting and the finishing time of the heating process; Tₑ and Tₐ are the tissue and the ambient temperatures, respectively, and q_{IMC} is the temperature correction factor.

Ultrasonic Measuring Channel (UMC)

Changes in the glucose concentration can be indirectly evaluated by the measurement of the velocity of sound propagating through the tissue. As the glucose concentration increases, the sound propagation velocity increases and the time during which the acoustic wave travels from one side of the body tissue to an opposite side of the body tissue decreases, as discussed in Zhao Z. Pulsed Photoacoustic Techniques and Glucose Determination in Human Blood and Tissue. Acta Univ. Oul C 169. Oulu, Finland, 2002; Toubal M, Asmani M, Radziszewski E, Nongaillard B. Acoustic measurement of compressibility and thermal expansion coefficient of erythrocytes. Phys Med Biol. 1999; 44:1277-1287; U.S. Pat. No. 5,119,819.

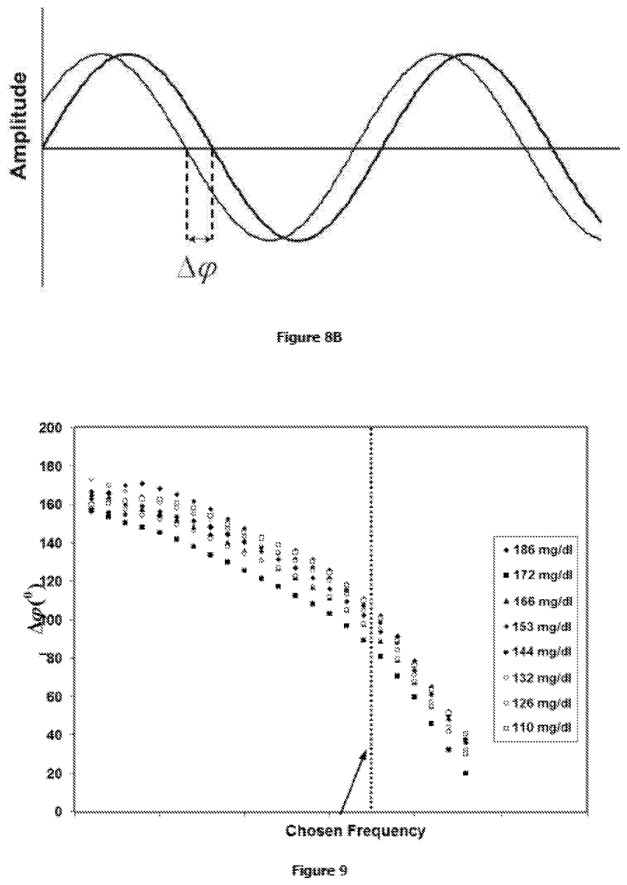

The UMC includes, in one example, of piezo transducers, specifically an ultrasonic transmitter 34 and an ultrasonic receiver 36, attached (or attachable) near the subject's ear lobe 16. Preferably, an electronic circuit is also provided for the ultrasonic measurement channel. The transmitter 34 (ultrasonic piezo transducer) is located in the external device (that is, the Sensor Unit 12), such that, when the external device is attached to the ear lobe, a continuous ultrasonic wave produced by the transmitter travels through the ear lobe with the characteristic velocity, causing a phase shift Δφ between the transmitted and received wave (FIG. 8B).

The piezo transducers—that is, the transmitter 34 and the receiver 36 (optionally followed by an amplifier)—are arranged one on each side of a subject's ear lobe (see, for example, FIG. 8A). The PU 10 sends a signal to the transmitter 34 to transmit a signal. After the transmitter 34 transmits the signal to the ear lobe 16, through which the transmitted signal propagates, the receiver 36 receives the propagated signal, steps up the received signal, and sends the stepped-up signal back to the PU 10 for processing with an algorithm to determine the corresponding value of the blood glucose concentration.

On opposing sides of the PEC, the piezo transducers—that is, the transmitter 36 and the receiver 34—are disposed. Generally, these ultrasonic transducers are sensitive to mechanical pressure. In order to protect the piezo transducers and to maintain the efficacy of the transducers, membranes 38 and 40 are preferably placed over the ultrasonic piezo transducers (see FIGS. 17 and 18). Preferably, an ultrasound-conductive adhesive or glue, such as epoxy, is placed between the membranes and the ultrasonic piezo transducers to hold the membranes firmly on the ultrasonic piezo transducers. Generally the adhesive or glue or epoxy should be suitable for conducting ultrasonic waves at a smallest signal loss. A layer of 0.05 mm is generally adequate for the adhering material.

The ultrasonic piezo transducers could be of any suitable size for being disposed in the PEC. In some examples, the transducers may be round and about 9.0 mm in diameter with less than 3.0 mm thickness in one example. The membranes 38, 40 may be made round and have a diameter of about 9.5 millimeters. It may be appreciated that any size is acceptable as long as it fits in the ear clip.

An electrically conductive and biocompatible coating is preferably placed on the outer surface of the membrane 38, 40 to enhance propagation of the signal. Typically, a coating of 0.01 mm is adequate.

The membranes may be made of nickel, which is generally biologically stable and conducts signals well. Any other suitable material, such as gold or titanium, may be used.

Preferably, the membranes 38, 40 are made of copper with a nickel coating. In an alternate embodiment, the membranes may be made of stainless steel and no coating would be needed.

In the preferred embodiment, it has been found that it is advantageous if one membrane 40 is flat and the other 38 is convex. This “hybrid” combination provides the best solution from a fitting point of view, and securely holds the device on the subject's ear lobe.

The ultrasonic wave's frequencies can range from 180 KHz to 10 MHz and transmitted amplitudes may vary from 0.5 V to 3 V in some examples. The received amplitudes may vary up to 50 mV. In some examples, the receiver amplifies the signal by about 20 times.

As illustrated in FIGS. 17 and 18, the ultrasonic piezo transducers preferably fit into the respective membranes with the adhesive (or epoxy) layer between them.

The velocity v of the acoustic wave's propagation through media and the monitored phase shift are related as follows.

$$v = 2 π \frac{fd}{Δ φ} \qquad ( 4 )$$

where f denotes the acoustic wave frequency (Hz); Δφ denotes the phase shift (radians); and d denotes the distance between piezo-transducers (m).

During calibration, two frequencies are identified and selected as optimal frequencies, one from a low-frequency range and one from a high-frequency range, where the frequency ranges are non-overlapping, as discussed in greater detail below with respect to a calibration procedure. After the calibration, the measurements are conducted at the two chosen frequencies.

FIG. 9 illustrates a graph of the measured phase-shift values as a family of functions having the frequency of excitation as an argument and the glucose concentration value as a parameter of the family.

This graph in FIG. 9 shows the phase shift versus input transducer frequency in the low-frequency region. The amplified phase shift values are viewed at the calibration-selected frequency value, which was found to be the optimal frequency during the calibration. That is, the calibration-selected frequency value is a frequency value that is determined during calibration to be an optimal frequency at which to perform phase shift measurements. Different curves on the graph apply to different glucose concentrations.

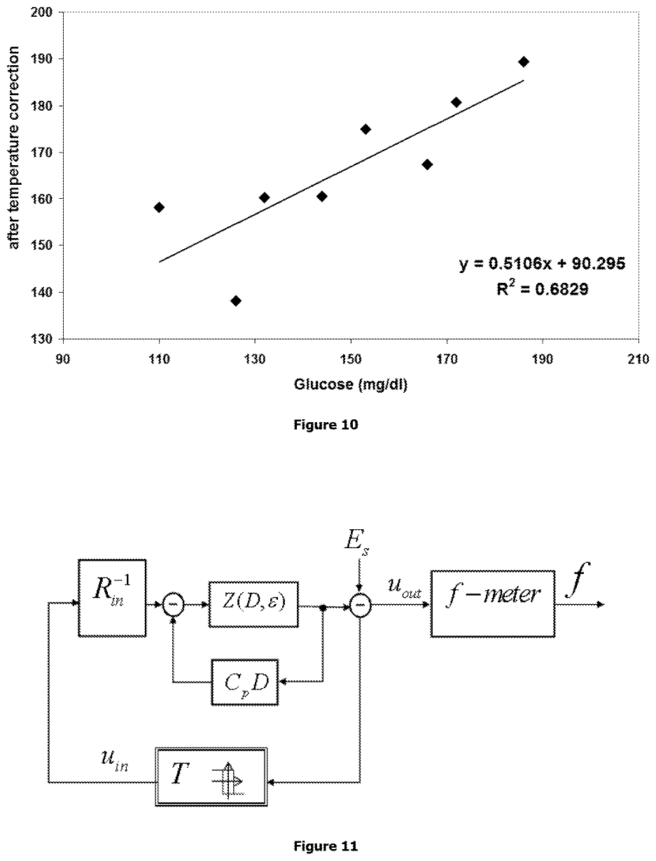

The velocity of the ultrasonic waves depends on the propagation medium temperature, as noted in U.S. Pat. No. 5,119,819; Zips A, Faust U. Determination of biomass by ultrasonic measurements. Appl Environ Microbiol. 1989 July; 55(7):1801-1807; Sarvazyan A, Tatarinov A, Sarvazyan N. Ultrasonic assessment of tissue hydration status. Ultrasonics. 2005; 43:661-671. The ambient temperature affects the sensor's parameters, whereas the tissue temperature impacts the wave's propagation in the tissue. Therefore, the temperature correction includes readings of both ambient and tissue temperature. The temperature correction is performed on the measured amplified phase shift signal u_{UMC}(t) (FIG. 10), using the following formula:

$${\hat{u}}_{UMC} ( t ) = {u}_{UMC} ( t ) - {q}_{UMC} ( 1 - \frac{{T}_{a}}{{T}_{e}} ) \qquad ( 5 )$$

where û_{UMC}(t) denotes the temperature-corrected amplified phase shift signal; and q_{UMC} denotes the correction factor. FIG. 10 is a graph showing the phase shift (measured at the chosen frequency) versus the BG with temperature correction enabled for a subject.

Electromangnetic Measuring Channel (EMC)

A glucose-induced water and ion transport across the cellular membrane leads to changes in the electrical properties of the cellular and consequently extracellular compartments, as indicated in Genet S, Costalat R, Burger J. The Influence of plasma membrane electrostatic properties on the stability of cell ionic composition. Biophys J. 2001 November; 81(5):2442-2457; Hayashi Y, Livshits L, Caduff A, Feldman Y. Dielectric spectroscopy study of specific glucose influence on human erythrocyte membranes. J Phys D: Appl Phys. 2003; 36:369-374. For example, a change in the dielectric properties is observed, as is known from Gudivaka R, Schoeller D, Kushner R F. Effect of skin temperature on multi-frequency bioelectrical impedance analysis. Appl Physiol. 1996 August; 81(2):838-845, which consequently is reflected in changes of the whole tissue impedance. To reflect changes in the tissue electrical impedance caused by varying glucose, the EMC includes a capacitor configured to be coupled to a user's body, such as the user's earlobe. For example, the capacitor may include two capacitor plates configured to be positioned on opposing sides of the user's earlobe, such that the user's earlobe acts as a dielectric material to the capacitor, as illustrated in FIG. 11. The EMC may further include a signal-generating component, such as an electromagnetic field generator, configured to generate and provide an oscillating signal to at least one plate of the capacitor, such that oscillating electromagnetic radiation is provided across the capacitor plates through the user's earlobe. In another example, the EMC may include an auto-oscillating circuit including two capacitor plates on opposite sides of a portion of a user's body, such as around the user's earlobe, where the portion of the user's body acts as a dielectric material in the auto-oscillating circuit. The capacitor may therefore be an active device or a passive device in certain examples. In various examples, properties of electromagnetic radiation between the capacitor plates, which reflect an impedance of a tissue of the user, may be analyzed to determine a glucose concentration of the user to whom the capacitor is coupled.

FIG. 11 shows the EMC wherein Rᵢₙ denotes the input resistance; Z(D,ε),

$$D ≡ \frac{d}{dt}$$

denotes the transfer operator of the sensing unit, which may include an EMC integrator including the earlobe tissue in the feedback; the transfer operator time constants depend on the tissue's electric permittivity denoted ε; Cₚ denotes the parasitic capacitance; f-meter is the auto-oscillation frequency (f) measuring circuit; T denotes the Relay unit with hysteresis creating a positive feedback in the auto-oscillating circuit; Eₛ denotes the electrical potential on the skin surface.

The same membranes 38 and 40 that were used in the UMC may preferably serve as capacitor plates having the earlobe 16 positioned between those plates as media with certain dielectric properties. An oscillator is used to generate signals and these signals depend on the parameters of the ear lobe. Frequencies may range from 5 kHz to 100 kHz and the amplitudes vary from about 0.1 V to 1.5 V.

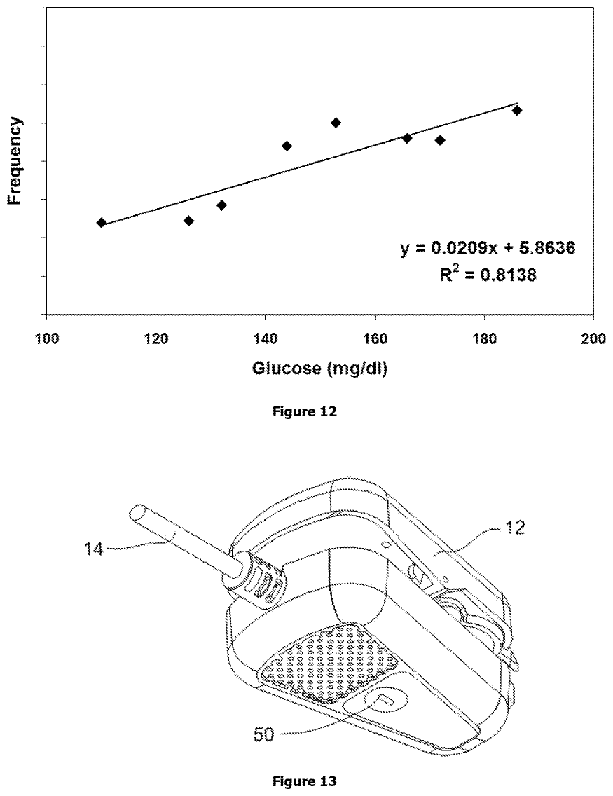

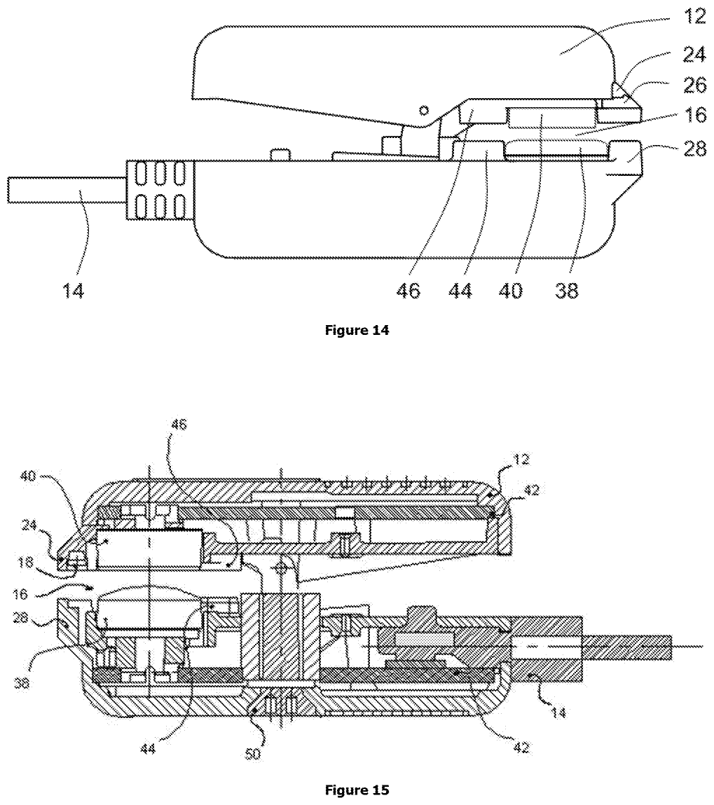

The earlobe temperature is also considered in the measurement, since the tissue's impedance is temperature-dependent, as discussed in Gudivaka R, Schoeller D, Kushner R F. Effect of skin temperature on multi-frequency bioelectrical impedance analysis. Appl Physiol. 1996 August; 81(2):838-845. Among the disturbance-representing variables of the EM Channel, the ambient temperature plays two roles: a) influencing the tissue parameters; b) affecting the sensor's electromagnetic parameters such as parasitic capacitance of electrodes. Therefore, the electromagnetic signal is corrected for both, ambient and ear temperatures, governed by the expression below and as illustrated in FIG. 12.

$${\hat{u}}_{EMC} = {u}_{UMC} ( t ) - {q}_{EMC} ( 1 - \frac{{T}_{a}}{{T}_{e}} ) \qquad ( 6 )$$

wherein û_{EMC} denotes the temperature-corrected EMC-generated signal proportional to the oscillating circuit's frequency; u_{UMC} denotes the non-corrected EMC-generated signal proportional to the oscillating circuit's frequency; and q_{EMC} denotes the correction factor.

In a preferred embodiment, there is also a distance sensor on the PEC. For example, the distance sensor may include a magnet 44 on one side of the PEC and a sensor 46 on the other side. The sensor 46, preferably a magnetic-field-measuring sensor, measures magnetic field intensity to ensure the distance between the membranes is the same as at a calibration stage.

FIG. 13 shows an example of the PEC. Preferably it is made of ABS plastic, but any suitable material will be effective. The size is dependent on the earlobe size of the subject. In a preferred embodiment, it is preferably about 25 mm long and about wide. It may be tapered. Preferably there will be different size clips to accommodate subjects of different sizes of earlobes.

As is well known for clips, preferably one side pivots about the other. One side has a pivot pin which fits into an appropriate seat in the other piece of the ear clip. A spring is used for biasing.

Preferably, an ambient temperature sensor 52 (FIG. 3) is also provided which may be located at the Sensor Unit 12, the PU 10, and/or may be placed on the cable 14.

Preferably, as is common in modern electronic devices, integrated circuit boards 42 are mounted within the ear clip 12 (FIG. 15). The aforesaid components of the three measuring sub-channels—ultrasonic, electromagnetic, and thermodynamic—are mounted on them. Then, either through the cable 14 or through wireless technology (such as Bluetooth), communication is established with the PU 10. As required, the PU 10 issues signals for activating each measurement channel and for then collecting data and thereafter calculating the blood glucose concentration value.

Calibration of the Apparatus

Preferably, there is a calibration performed prior to glucose measurements, so that the influence of individual quasi-stable factors, such as tissue structure, can be minimized. The sensor is individually adjusted for optimal fit (PEC Positioning Adjustment Step), according to the thickness of the subject's earlobe, prior to calibration. Preferably the adjustment screw 50 (FIGS. 4, 13, and 15) is used to adjust the distance between the sensors and consequently the pressure on the earlobe for optimal fit. This action may be guided by the PU 10. The optional distance sensor 44, 46 preferably assures this preset distance is maintained.

After adjusting the PEC, the calibration process begins. One preferred procedure for calibration is set forth herein.

The calibration procedure associates invasive basal and post-prandial blood glucose data (taken from the capillary blood of a fingertip and measured by a reference invasive device) with six sequential measurements timely produced by the apparatus and builds a calibration curve unique to each individual.

The first three calibration points are performed at the same (fasting) glucose concentration and help establishing an accurate initial point for the model used in the calibration. They are performed in the fasting state, consisting of one invasive and three consecutive non-invasive measurements, followed by food and drink consumption, in order to increase blood glucose by at least 30% from the fasting value. 20 minutes post-meal, a set of five sequential measurement pairs, with time intervals of about 10 minutes in between is taken. In total, the calibration process takes about 1.5 to 2 hours.

At the first point of the calibration, the distance is automatically measured (by means of the optional distance sensor 44, 46 provided in the PEC or by using an alternative method) and set as a reference distance (original location or preset reference point) of the sensors, which, in the following calibration points, as well as measurement points will be checked, prior to beginning the measurements. The earlobe is a mostly parallel fiber tissue with a smooth surface. Therefore, if the distance in any of the calibration points, or in a regular measurement points differs (within a certain tolerance range) from the preset reference point, the user is guided by the device to move the PEC as required, in order to get to the reference distance. Once the calibration is completed, a vector of an individual linear model's parameters is set for each technology's output.

For the thermodynamic technology, the heating intensity (combined thermal diffusivity of the heating members) is checked during the measurement of the first point and the correction factor is calculated for the optimal heating intensity, to be used in the consequent measurements. This factor is individually calculated for each user, in order to assure increasing the tissue surface temperature above a minimal increment threshold.

For the electromagnetic technology, the oscillations are performed at a certain frequency, which is found as a function of said frequency sensitivity to the BG changes during the calibration.

In order to choose optimal working frequencies for the acoustic measurement method, a sweeping of two frequencies' regions is performed in low- and high-frequency regions during calibration. In each region, the optimal frequency is selected, according to the signal's amplitude (the strength of the propagated signal) and the sensitivity of the phase shift to glucose changes at that particular frequency. That is, an optimal frequency is a frequency at which an amplitude of the signal and the sensitivity of the phase shift to glucose changes is maximized. After the calibration, the measurements are performed at these two “calibration-selected” optimal frequencies (one from the low region and one from the high region).

According to another example, either the ambient temperature or the tissue temperature is acquired for being used in the temperature correction links of the device.

After the calibration, the BG spot measurements can be performed by clipping the PEC to the earlobe for the duration of the measurement (about one minute) and removing it afterwards.

After automatically confirming the correct sensor's positioning against the distance reference established during the PEC Positioning Adjustment Step, the measurement begins. Each measuring channel produces several outputs, upon which a signal validation and recognition of outliers is automatically applied.

For the signal validation of the UMC, the signal's amplitude for each selected frequency is checked, to ensure the proper wave propagation through the tissue.

Because the members of the EMC and TMC channels are physically mounted on the same area on the tissue, the low measured amplitudes indicate a poor-quality mechanical contact. In this case the measurement is disregarded, and the failure notice is provided to the user. In the TMC, the sensing thermistor within the PEC is mounted on a different tissue area than the electromagnetic and ultrasonic sensors. Therefore, a good-quality mechanical contact for the two latter technologies does not provide for the same for the thermodynamic channel. Thus, the heating process is checked for the allowed temperature interval. The out-of-range temperature signal is regarded as the product of the poor quality of the mechanical contact and a failure notice is provided to the user.

In one example, the received BG values from each measuring channel are used to generate the final measurement in accordance with the linear combination paradigm.

In another embodiment example, the readings from the measuring channels are analyzed based on the directions of their respective trendlines. Subsequently, weights are assigned to each of the three measuring channels' outputs as described in Freger. Finally, a weighted linear combination of said three outputs produces a more precise BG reading than each participating measuring channel does if said measuring channels are characterized by the identical precision.

The precision value of each said participating measuring channel lies approximately within ±10% of the precision values of other participating measuring channels. Accordingly, examples provided herein converge to the system of expression (1), above.

Non-invasive BG measuring devices perform their measurements by sensing physiological phenomena that develop within the tissue due to changes occurring with the BG. Because those physiological processes are affected by multiple factors unrelated to the BG factors, such as ambient temperature, the outcome of each non-invasive BG measurement carries errors.

In part, the above-described temperature corrective techniques were designed to lower the adverse effect of the ambient and the earlobe-tissue temperatures by making the device's data processing during each instantaneous measurement correspond with the environmental conditions existing at the time of calibration.

To further minimize those errors, another configuration of the apparatus combining multiple measurement channels that evaluate the BG estimating variables of different physical nature is suggested. Each measuring channel trends the BG because its calibration curve uniquely links some tissue property's accessible physical variable (that is, the calibration curve's input) to the BG as its output. However, each measuring channel measures under the same set of disturbing factors. As was shown above, the simultaneous evaluation of the outputs of those measuring channels allows the improvement of the instrument's performance and, in particular, the precision.

However, the idea of the simultaneous or sequential evaluation of outcomes of multiple measuring channels to implement a better-performing non-invasive BG measuring apparatus carries an additional benefit directly improving another statistical characteristic of the measurement instrument's performance—the accuracy of the measurement.

The linear combination of outcomes of multiple correctly trending MCs of similar precision utilizing different (non-identical) BG-informative physical variables will deliver the improved measurement precision but will not protect from non-random dependencies on the disturbing variables like ambient temperature. This non-random influence adversely affects the accuracy of the BG measurement.

To simultaneously improve the precision and the accuracy of the BG measurement, it is suggested in the present invention to jointly use multiple measuring channels monitoring unique-per-measuring-channel BG-informative physical variables with at least two of said variables possessing the property of orthogonality toward the disturbing variable or variables common in those measuring channels.

More particularly, in one example, with regard to the EMC, it reflects changes in the tissue's electrical impedance caused by the varying glucose concentration. The EMC produces electrical current oscillations which period κ depends on the electrical capacitance C of the space between the ear clip membranes having a human's earlobe between them. The capacitance depends on the values of the dielectric constant of the earlobe tissue ε, which is proportional to the glucose concentration, and also on the so-called parasitic capacitance of the ear clip. Therefore, the blood glucose concentration can be represented by the following expression:

BG_{EMC}=f{τ[ε,ξ_{EMC}(Tₐ)]}, (7)

wherein BG_{EMC} denotes the blood glucose concentration measured by the EMC; and ξ_{EMC} denotes a vector of parameters of the EMC unrelated to glucose concentration. Those parameters characterize the mechanical and electrical construction of the ear clip and are the subjects to the disturbing effect of varying ambient temperature. Because the healthy human body temperature does not vary significantly as opposed to the ambient temperature, the dielectric constant of the earlobe tissue ε is not affected by the ambient temperature variations.

The most sensitive to ambient temperature part of the EMC is the area of the mechanical contact between the ear clip membrane and the earlobe tissue. The tissue's sweat glands produce conductive secretions in relation with the ambient temperature Tₐ. The accumulation of the sweat glands' secretion produces an effect commonly known as the “Supercapacitor” effect, thereby lowering the κ, thereby increasing the measured value of the BG. The EMC-generated BG value can be approximated by the following expression:

$$B {G}_{EMC} ( e , {T}_{a} ) \approx {BG}_{EMC}^{*} ( e , {T}_{a}^{*} ) + {a}_{EMC} ( {T}_{a} - {T}_{a}^{*} ) , \\ {a}_{EMC} = \frac{\partial }{\partial {T}_{a}} {{BG}_{EMC} ( e , {T}_{a} )}_{\vert {T}_{a} = {T}_{a}^{*}} > 0 \qquad ( 8 )$$

wherein, BG_{EMC}′(ε) denotes the true (undisturbed) glucose concentration measured via the EMC, and Tₐ* denotes the reference value of the ambient temperature, for example, the ambient temperature obtained at the time of calibration. The a_{EMC} is the coefficient of the second term of the Taylor series.

With regard to the UMC, a continuous ultrasonic wave produced by the piezo transducer travels through the ear lobe with characteristic velocity, causing a phase shift between the transmitted and received wave. The velocity v and the phase shift are related:

$$Δϕ = \frac{2 π fd}{v} \qquad ( 9 )$$

where f is the frequency (Hz) of the ultrasonic wave; Δφ is the phase shift (radians) between the transmitted and received ultrasonic wave; and d is the distance between piezo-transducers of the sensors (m). The UMC measures glucose concentration by monitoring the phase shift Δφ between the sent and the received ultrasonic wave traveling through the earlobe. The path of the ultrasonic wave has several regions and primarily includes the region between the piezo transducer emitting the ultrasonic wave at one end of the earlobe and the earlobe epidermis, earlobe-tissue region, and the region between the epidermis on other side of the earlobe and the piezo transducer receiving the generated ultrasonic wave. The part of the phase shift contributed by the tissue region depends on the tissue's compressibility, which depends on the glucose concentration: BG_{UMC}*=BG_{UMC}*(γ,μ), where BG_{UMC}* denotes undisturbed output of the UMC, γ denotes the glucose compressibility; and μ denotes the glucose viscosity. It is further known that the increase in glucose concentration can be associated with the decrease in the monitored phase shift between the sent and received ultrasonic wave, as discussed above.

It follows from the design of the ear clip that the ambient temperature variations have a strong effect on the monitored phase shift. The ear clip's main mechanical parameter reacting to changes in the ambient temperature is the spring-controlled distance d between the transducers. Changes in the ambient temperature cause negatively correlated changes in the stiffness of the spring, such that increases in temperature reduces the stiffness of the spring. Due to mechanical reaction forces from the earlobe, the lower the ear clip spring stiffness, the more the distance d increases, causing the phase shift to increase. The phase shift increase is perceived by the UMC as the decrease in the blood glucose value.

Based on the above, the output of the UMC can be described as follows:

BG_{UMC}=f{φ[γ,μ,ξ_{UMC}(Tₐ)]} (10)

Wherein, ξ_{UMC} denotes a vector of parameters of the UMC that are unrelated to glucose concentration; d∈ξ_{UMC}.

Accordingly, The UMC-generated BG value can be approximated by the following expression:

$${BG}_{UMC} ( γ , μ , {T}_{a} ) \approx {BG}_{UMC}^{*} ( γ , μ , {T}_{a}^{*} ) - {a}_{UMC} ( {T}_{a} - {T}_{a}^{*} ) , \\ {a}_{UMC} = \vert \frac{\partial }{\partial {T}_{a}} {{BG}_{UMC} ( γ , μ , {T}_{a} )}_{\vert {T}_{a} = {T}_{a}^{*}} \vert > 0 \qquad ( 11 )$$

If the glucose concentration measured by each measuring channel has the same units of measurement, a linear combination of the outputs of the EMC and UMC explicitly demonstrates the reduction of the negative effect of the ambient temperature on the performance of the multi-channel measuring device.

$$\begin{matrix}BG = f ( {BG}_{EMC} + B {G}_{UMC} ) \\ = [ {BG}_{EMC}^{*} ( ɛ , {T}_{a}^{*} ) + {a}_{EMC} ( {T}_{a} - {T}_{a}^{*} ) + \\ {BG}_{UMC}^{*} ( γ , μ , {T}_{a}^{*} ) - {a}_{UMC} ( {T}_{a} - {T}_{a}^{*} ) ]\end{matrix} \qquad ( 12 )$$

Because the BG_{EMC}*(ε) and BG_{UMC}*(γ,μ) are both the true measurements of the glucose concentration, the formula (12) can be simplified as follows:

$$\begin{matrix}BG = f ( {BG}_{EMC} + {BG}_{UMC} ) \\ = {BG}^{*} + 0.5 ( {a}_{EMC} - {a}_{UMC} ) ( {T}_{a} - {T}_{a}^{*} )\end{matrix} \qquad ( 13 )$$

because

(a_{EMC}>0)∧(a_{UMC}>0)⇒|a_{EMC}−a_{UMC}|<(a_{EMC}∨a_{UMC}) (14)

Accordingly, it follows from the formula (14), that its output is always closer to the true glucose value than the output of either the EMC (8) or the UMC (11), wherein the determined measurement of the glucose concentration more accurately represents the glucose concentration than any one of the measurements respectively received from the measuring subchannels making an orthogonal pair.

The same reasoning can be applied to a combination of outputs of another pair of measurement subchannels having the TMC as the pair's component. According to expression (3), the TMC measures the amount of energy required to increase the heater's temperature to a certain value over a given period of time. To rephrase, the TMC evaluates the measuring system's thermal diffusivity (α):

$$α = \frac{k}{{pC}_{p}}$$

Where k denotes the material thermal conductivity, ρ denotes the material density, and Cₚ denotes the material specific heat. Within the scope of the present invention, the material is a complex system comprised of the sensing clip and the earlobe tissue, thus

α=α(BG,k_{T},ρ_{T},C_{pT},ξ_{TMC}(Tₐ)),

k_{sg}∈ξ_{TMC}(Tₐ) (15)

Where ξ_{TMC}(Tₐ) denotes the vector of the TMC parameters unrelated to the BG, k_{sg} denotes the thermal conductivity of the earlobe sweat glands' secretion, and k_{T}, ρ_{T}, C_{pT} denote the thermal conductivity, density, and specific heat of the tissue respectively.

According to the construction of the TMC, an attachment of the ear clip to the earlobe disturbs the thermodynamic equilibrium, thereby causing sweat glands to increase the production of their secretions and filling voids between the membrane and the earlobe epidermis, which increases the thermal conductivity of the area of contact between the clip membrane and the earlobe tissue. This results in the increase of the entire thermodynamic system's thermal diffusivity which will be perceived by the TMC as the erroneous increase of the monitored BG:

$${BG}_{TMC} = B {G}_{TMC}^{*} + {a}_{TMC} ( {T}_{a} - {T}_{a}^{*} ) , \\ {a}_{TMC} = \frac{\partial }{\partial {T}_{a}} {{BG}_{TMC} ( α , {T}_{a} )}_{\vert {T}_{a} = {T}_{a}^{*}} > 0 \qquad ( 16 )$$

Thus, a pair of measuring channels with orthogonal properties toward the disturbing ambient temperature including the TMC and the UMC will produce readings with higher accuracy than each of the MC comprising the pair:

$$\begin{matrix}BG = f ( {BG}_{TMC} + {BG}_{UMC} ) = \\ = f [ {BG}_{TMC}^{*} ( α , {T}_{a}^{*} ) + {a}_{TMC} ( {T}_{a} - {T}_{a}^{*} ) + \\ {BG}_{UMC}^{*} ( γ , μ , {T}_{a}^{*} ) - {a}_{UMC} ( {T}_{a} - {T}_{a}^{*} ) ] ,\end{matrix} \\ BG = {BG}^{*} + 0.5 ( {a}_{TMC} - {a}_{UMC} ) ( {T}_{a} - {T}_{a}^{*} ) , \\ ( {a}_{TMC} > 0 ⋀ {a}_{UMC} > 0 ) ⇒ 0.5 \vert {a}_{TMC} - {a}_{UMC} \vert < ( {a}_{TMC} ⋁ {a}_{UMC} ) \qquad ( 17 )$$

It follows from the expressions (13), (14), and (17) that configuring measuring channels as pairs of subchannels with orthogonal properties toward the common disturbance is beneficial with regards to the accuracy of the BG measuring apparatus. The linear combination part of the information structure for the above example can be illustrated with the following example, in which a true value of the BG being measured is 90 mg/dL. In this example, suppose that instantaneous BG readings of the TMC, EMC, and UMC are:

BG_{EMC}=90+18 mg/dL; BG_{TMC}=90+10 mg/dL; BG_{UMC}=90−15 mg/dL.

In certain existing devices, such as that of Freger, a final measurement may be determined as a linear combination of the three measurements, that is, ⅓(BG_{TMC}+BG_{EMC}+BG_{UMC})=94.33 mg/dL. In this example, an instantaneous absolute measurement error for Freger's method is 4.33 mg/dL. Conversely, implementing the above-described examples of the present disclosure where UMC and EMC form a first subchannel and UMC and TMC form a second subchannel yields a final measurement of, ¼ (BG_{UMC}+BG_{EMC}+BG_{UMC}+BG_{TMC})=90.25 mg/dL. Accordingly, a reduced measurement error may be achieved by using pairs of orthogonal subchannels, at least because as a disturbance variable (for example, temperature) increases a BG measurement of one subchannel in a pair (for example, the EMC or TMC subchannels), the disturbance variable decreases a BG measurement of another subchannel in a pair (for example, a UMC subchannel) such that errors induced by the disturbance variable are canceled out between the pair's measurements.

Therefore, an application of the moving average procedure to the linear combination of the number of measuring channels created by adding outputs of pairs of measuring sub-channels (13), and (17) creates a configuration of the BG measuring apparatus with the improved accuracy and precision if compared with the accuracy and precision of each individual sub-channel. The linear combination algorithm can be based on the weighted or non-weighted paradigm and the gain values of measuring subchannels in each measuring channel can be selected based on the peculiarities of the measuring apparatus implementation.

In the generalized form, the information block diagram of FIG. 2 (which may represent an information block diagram implemented by devices discussed herein) and an associated data-processing algorithm are presented in the following expression in the style of expression (1).

$$\overline{u ( t )} = {m}^{- 1} {\sum }_{i = 1}^{m} \frac{1}{2 n} {\sum }_{q = 1}^{n} ( {x}_{q , 1 , i} + {x}_{q , 2 , i} ) , \\ {x}_{q , r , i} ≡ {{x}_{q , r} ( t )}_{\vert t = {t}_{i}} , \\ {x}_{q , r} ( t ) = {G}_{q , r} {W}_{q , r} ( D , {u}_{q , r} ( t ) , {ξ}_{q , r} ( t ) ) , \\ m > 1 , n \geq 1 ; r = 1 , 2 ; D ≡ \frac{d}{d t} \qquad ( 18 )$$

Where x_{q,r,i} denotes the i^{th} instance (t=tᵢ) of the r^{th} measuring subchannel constituting the q^{th} measuring channel; G denotes the controlled gain of a measuring subchannel and the W(D,u(t),ξ(t)) denotes the measuring subchannel's transfer operator; n denotes the number of measuring channels comprised of pairs of subchannels.

Unless a priori knowing that the performance of the particular measurement sub-channel from the selected group of measurement channels is substantially superior than the performance of the remaining members in the group, the suggested structure of the BG measuring instrument will allow measuring the BG with increased performance for both the spot and the continuous measurement applications.

To practice the method of the present invention, it may be sufficient to repeat calibration over the time because of the quasi-stationary nature of the human tissue and environmental properties.

Although in certain examples measuring channels may include two subchannels, in other examples, measuring channels may include a single subchannel. For example, while certain measurement channels may include TMC and UMC and/or EMC and UMC, in other examples, measurement channels may include only one of TMC, EMC, and UMC. A single BG measuring device may include several measurement channels, where each measurement channel may include one or more subchannels of the same or a different type, that is, TMC, EMC, or UMC. It is to be appreciated that each of the TMC, UMC, and EMC may be referred to as a subchannel.

It is to be appreciated that, although certain device configurations have been illustrated as examples, alternate examples are within the scope of the disclosure. For example, although certain examples may include a PU 10 and a Sensor Unit 12, it is to be appreciated that alternate devices may be provided to execute certain functions. For example, certain devices may include one or more sensors (for example, disposed in, on, or in connection with, the Sensor Unit 12) configured to measure certain physical parameters, such as temperature, electromagnetic radiation, and/or ultrasonic waves, and one or more controllers coupled to the sensor(s). The one or more sensors may further include components acting in concert with physical-parameter-measuring components. For example, the one or more sensors may include a heater and a heat sensor, a capacitor and an auto-oscillator, and/or one or more ultrasonic piezo transducers. The controller(s) may receive information from the sensor(s) indicative of the physical variables, such as raw measurement data, and process the data pursuant to the examples provided above to yield a BG value. In other examples, a device may include one or more intermediate components between the sensor(s) and the controller(s) to at least partially modify or process the measurement data provided by the sensor(s) prior to providing the measurement data to the controller(s). For example, a device may include filtering components configured to filter or otherwise modify the measurement data prior to providing the modified measurement data to the controller(s) for further processing. The device may include a controller configured to operate in a manner consistent with the information flow of FIG. 2, where the controller receives measurement data from measurement channels having pairs of orthogonal subchannels and determines a final BG value based on information received from the measurement channel(s).

While certain features of the invention have been illustrated and described herein, many modifications, substitutions, changes, and equivalents will now occur to those of ordinary skill in the art. The invention is described in detail with reference to a particular embodiment, but it should be understood that various other modifications can be effected and still be within the spirit and scope of the invention. It is, therefore, to be understood that the appended claims are intended to cover all such modifications and changes as fall within the true spirit of the invention.

We claim:

1. An apparatus for non-invasively measuring a blood glucose concentration in a subject, the apparatus comprising:
a plurality of sensors, each sensor of the plurality of sensors being configured to determine sensor information indicative of a respective physical variable of a plurality of physical variables, each respective physical variable being indicative of the blood glucose concentration in the subject; and
a controller coupled to the plurality of sensors, the controller being configured to:
receive respective sensor information from each sensor of the plurality of sensors;
determine at least one measurement channel each including an orthogonal pair of sensors from the plurality of sensors, each orthogonal pair of sensors including a first sensor to determine first sensor information indicative of the blood glucose concentration of the subject and a second sensor to determine second sensor information indicative of the blood glucose concentration of the subject, wherein the orthogonal pair of sensors is orthogonal with respect to a disturbance variable that induces a positive error in one of the first sensor information and the second sensor information and that induces a negative error in the other of the first sensor information and the second sensor information; and
determine a blood glucose measurement of the subject based on the first sensor information and the second sensor information.

2. The apparatus of claim 1, wherein the at least one orthogonal pair of sensors includes a single pair of sensors.

3. The apparatus of claim 1, wherein the disturbance variable includes an ambient temperature.

4. The apparatus of claim 1, wherein the plurality of physical variables includes one or more of:
a property of an ultrasonic wave propagating through a tissue of the subject;
a property of an electromagnetic impedance of the tissue of the subject; and
a property of a heatwave propagating through the tissue of the subject.

5. The apparatus of claim 4, further comprising a housing including the plurality of sensors and a fastener configured to affix the housing to the subject.

6. The apparatus of claim 5, wherein the plurality of sensors includes a first ultrasonic piezo transducer and a second ultrasonic piezo transducer, and wherein the first ultrasonic piezo transducer and the second ultrasonic piezo transducer are configured to be coupled to opposing sides of a portion of a body of the subject.

7. The apparatus of claim 6, further comprising a respective membrane covering each of the first ultrasonic piezo transducer and the second ultrasonic piezo transducer.

8. The apparatus of claim 7, wherein at least one of the first ultrasonic piezo transducer includes an ultrasonic-wave transmitter and the second ultrasonic piezo transducer includes an ultrasonic-wave receiver, wherein the first ultrasonic piezo transducer is configured to transmit an ultrasonic wave to the body of the subject, and wherein the second ultrasonic piezo transducer is configured to receive the ultrasonic wave from the body of the subject.

9. The apparatus of claim 8, wherein the first sensor includes the first ultrasonic piezo transducer and the second ultrasonic piezo transducer, and wherein the first sensor information includes a phase shift between the transmitted ultrasonic wave and the received ultrasonic wave.

10. The apparatus of claim 5, wherein the plurality of sensors includes a first capacitor plate, a second capacitor plate, and an auto-oscillator configured to generate an oscillating signal between the first capacitor plate and the second capacitor plate, and wherein the first capacitor plate and the second capacitor plate are configured to be positioned on opposing sides of a portion of a body of the subject.

11. The apparatus of claim 10, further comprising a respective membrane covering each of the first capacitor plate and the second capacitor plate.

12. The apparatus of claim 11, wherein the first sensor includes the first capacitor plate and the second capacitor plate, and wherein the first sensor information includes a tissue impedance of the subject.

13. The apparatus of claim 5, wherein the plurality of sensors includes a heater and a heat sensor configured to be coupled to a portion of a body of the subject.

14. The apparatus of claim 13, wherein the first sensor includes the heater and the heat sensor, and wherein the first sensor information includes heat transfer characteristics of the subject.

15. The apparatus of claim 5, wherein the fastener includes opposing sides configured to affix the housing to a body of the subject, the apparatus further comprising at least one distance sensor configured to measure a distance between the opposing sides of the fastener.

16. The apparatus of claim 15, wherein the at least one distance sensor includes a magnet and a magnetic-field sensor.

17. The apparatus of claim 16, further comprising an adjustment screw configured to set the distance between the opposing sides of the fastener.

18. The apparatus of claim 4, wherein the at least one measuring channel includes a first measuring channel and a second measuring channel, the first measuring channel including a first orthogonal pair of sensors, the first orthogonal pair of sensors being configured to measure a first and a second of the property of the ultrasonic wave propagating through the tissue of the subject, the property of the electromagnetic impedance of the tissue of the subject, and the property of the heatwave propagating through the tissue of the subject, and the second orthogonal pair of sensors being configured to measure the first and a third of the property of the ultrasonic wave propagating through the tissue of the subject, the property of the electromagnetic impedance of the tissue of the subject, and the property of the heatwave propagating through the tissue of the subject.

19. A method for non-invasively measuring a blood glucose concentration in a subject, the method comprising:
determining, with each sensor of a plurality of sensors, sensor information indicative of a respective physical variable of a plurality of physical variables, each respective physical variable being indicative of the blood glucose concentration in the subject;
determining at least one measurement channel each including an orthogonal pair of sensors from the plurality of sensors, each orthogonal pair of sensors including a first sensor to determine first sensor information indicative of the blood glucose concentration of the subject and a second sensor to determine second sensor information indicative of the blood glucose concentration of the subject, wherein the orthogonal pair of sensors is orthogonal with respect to a disturbance variable that induces a positive error in one of the first sensor information and the second sensor information and that induces a negative error in the other of the first sensor information and the second sensor information; and
determining a blood glucose measurement of the subject based on the first sensor information and the second sensor information.

20. A system for non-invasively measuring a blood glucose concentration in a subject, the system comprising:
a plurality of sensors, each sensor of the plurality of sensors being configured to determine sensor information indicative of a respective physical variable of a plurality of physical variables, each respective physical variable being indicative of the blood glucose concentration in the subject; and
means for determining a blood glucose measurement of the subject based on sensor information from at least one orthogonal pair of sensors more accurately and more precisely than any individual sensor of the at least one orthogonal pair of sensors.
